(19) United States
(12) Patent Application Publication
(10) Pub. No.: US 2010/0280093 A1
(43) Pub. Date: Nov. 4, 2010

(54) PROCESS FOR THE PREPARATION ENANTIOMERICALLY PURE SALTS OF N-METHYL-3-(1-NAPHTHALENEOXY)-3-(2-THIENYL)PROPANAMINE

(75) Inventors: Sujoy Biswas, West Bengal (IN); Keya Karanjai, Uttar Pradesh (IN); Chandra Has Khanduri, Gurgaon (IN)

Correspondence Address: Ranbaxy Inc., Intellectual Property Department, 600 College Road East, PRINCETON, NJ, 08540, US

(73) Assignee: RANBAXY LABORATORIES LIMITED, Haryana (IN)

(21) Appl. No.: 12/305,766

(22) PCT Filed: Jul. 3, 2007
(86) PCT No.: PCT/IB2007/052604

§ 371 Date: Jul. 15, 2010

(30) Foreign Application Priority Data
Jul. 3, 2006 (IN) 1553/DEL/2006

(51) Int. Cl. A61K 31/381; C07D 333/20; A61P 25/24

(52) U.S. Cl. 514/438; 549/75

(57) ABSTRACT

The present invention relates to duloxetine salts having enantiomeric purity of 98% or more and a process for such salts.

### FIELD OF THE INVENTION

The present invention relates to a process for the preparation of duloxetine and its salts.

### BACKGROUND OF THE INVENTION

Duloxetine hydrochloride is a selective serotonin and norepinephrine reuptake inhibitor (SSNRI) for oral administration. It is chemically (+)-(S)-N-methyl-γ-(1-naphthyloxy)-2-thiophenepropylamine hydrochloride as represented by Formula I:

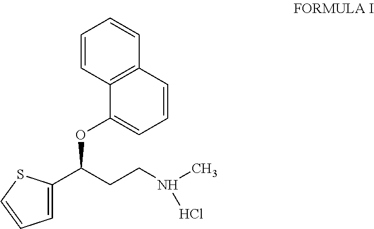

U.S. Pat. No. 5,023,269 (the '269 patent) provides a process for the preparation of racemic N-methyl-γ-(1-naphthyloxy)-2-thiophenepropylamine oxalate. Though the '269 patent discloses the maleate and oxalate salts of S-(+)-N-methyl-γ-(1-naphthyloxy)-2-thiophenepropylamine, it does not disclose any method to prepare the two enantiomers of N-methyl-γ-(1-naphthyloxy)-2-thiophenepropylamine or their salts. Tetrahedron Letters 1990, 31(49), 7101-7104 provides a process for preparing duloxetine by dealkylating the oxalate salt of the compound of Formula II. In this process, the final compound of duloxetine is isolated as oxalate or maleate salt.

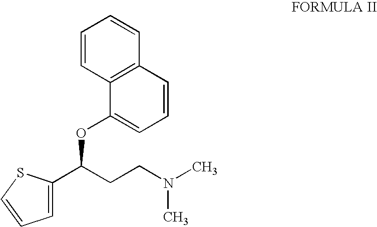

U.S. Pat. No. 5,491,243 (the '243 patent) provides a process for preparing duloxetine, wherein the phosphoric acid salt of the compound of Formula is used as an intermediate, which is demethylated to obtain duloxetine. The '243 patent mentions that the phosphoric acid salt of the compound of Formula II, which is the penultimate intermediate, has a purity of 91% EE. In this process, the final compound of duloxetine is isolated as a hydrochloride salt after demethylation.

The processes for the preparation of duloxetine and its intermediates are also provided in EP 0,457,559 A3, U.S. Pat. No. 5,362,886, WO 03/062219, WO 03/070720, EP 1,506,965, WO 04/005307, US 2004/0181058, WO 04/056795, WO 04/065376, WO 04/055194, WO 03/018572, JP 2003-192681 A2, US 2003/225153, US 2005/107621, WO 04/005220, WO 04/005239, WO 04/011452, WO 04/013123, WO 04/016603, DE 10237272 A1, WO 04/020389, WO 04/024708, WO 04/031168, EP 1411045 A1, DE 10248479 A1, DE 10248480 A1, WO 04/090094, WO 04/103990, WO 05/021527, WO 05/033094, WO 05/073215, WO 05/080370, US 2003/225274, US 2003/225153, US 2004/023348, US 2004/023344, U.S. Pat. No. 6,924,386, DE 10237272 A1, and US 2004/181058.

### SUMMARY OF THE INVENTION

The present inventors have observed that the reported processes for preparing duloxetine using the compound of Formula II as an intermediate require the isolation and purification of the intermediates at various stages to obtain the final compound. Specifically, the prior art processes involve the isolation of compound of Formula II as a salt of phosphoric acid or oxalic acid.

The present inventors have developed a process for the preparation of duloxetine and its salts wherein isolation and purification of all intermediates is not needed. Further, the present invention provides a process for the preparation of duloxetine and its salts with high enantiomeric purity directly from the free base of the compound of Formula II, while reducing the processing steps involved in the salt formation and isolation of the compound of Formula II. Thus, the present process is simple, economic and industrially preferable for preparing duloxetine and salts.

### DETAILED DESCRIPTION OF THE INVENTION

In one aspect of the present invention is provided a process for the preparation duloxetine of Formula I or its salts,

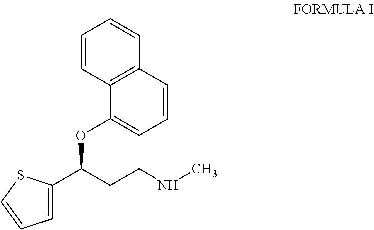

wherein the process comprises,
a) reacting (1S)-3-(dimethylamino)-1-(2-thienyl)propan-1-ol of Formula III or its salts,

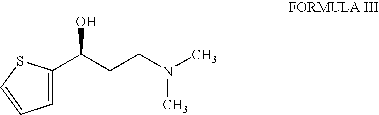

with 1-fluoronaphthalene in an organic solvent to obtain (3S)-N,N-dimethyl-3-(1-naphthyloxy)-3-(2-thienyl)propan-1-amine of Formula II or its salts,

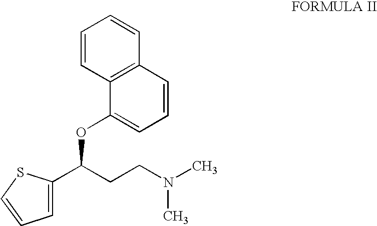

b) dealkylating (3S)-N,N-dimethyl-3-(1-napthyloxy)-3-(2-thienyl)propan-1-amine of Formula II or its salts to obtain duloxetine of Formula I or its salts, and
c) isolating duloxetine of Formula I or its salts from the reaction mixture thereof, wherein the compound of Formula II need not be isolated from the reaction mixture in any solid form, including in the form of a salt.

A another aspect of the present invention is provided a process for the preparation duloxetine of Formula I or its salts,

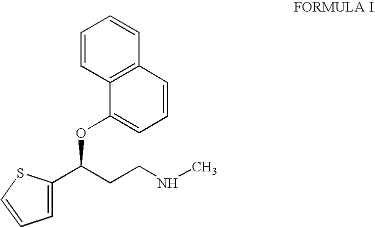

wherein the process comprises,
a) reacting (1S)-3-(dimethylamino)-1-(2-thienyl)propan-1-ol of Formula III or its salts;

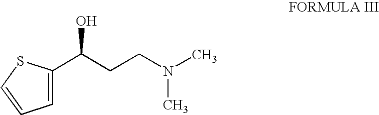

with 1-fluoronaphthalene in an organic solvent to obtain (3S)-N,N-dimethyl-3-(1-naphthyloxy)-3-(2-thienyl)propan-1-amine of Formula II as a free base,

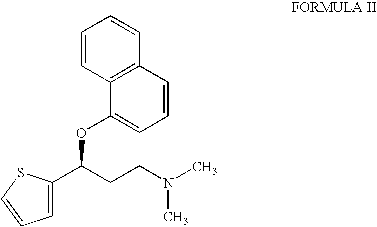

b) dealkylating the free base of (3S)-N,N-dimethyl-3-(1-naphthyloxy)-3-(2-thienyl)propan-1-amine of Formula II to obtain duloxetine of Formula I or its salts, and
c) isolating duloxetine of Formula I or its salts from the reaction mixture thereof.

(1S)-3-(dimethylamino)-1-(2-thienyl)propan-1-ol of Formula III or its salts can be prepared according to the methods provided in U.S. Pat. No. 5,491,243 or according to the method disclosed in the present application. The compound of Formula III as a free base or in any salt form is reacted with 1-fluoronaphthalene in the presence of an organic solvent to obtain (3S)-N,N-dimethyl-3-(1-naphthyloxy)-3-(2-thienyl)propan-1-amine of Formula II or its salts. The organic solvent is selected from the group consisting of dimethylsulfoxide, C₁₋₃ alkanol, toluene, chloroform, dioxane, dimethylformamide, dimethylacetamide, and tetrahydrofuran. The organic solvent is more preferably dimethylsulfoxide or dimethylacetamide. The compound of Formula II can be obtained in the form of a free base, which need not be isolated from the reaction mixture in any solid form, including as a salt. The compound of Formula II can be dealkylated using phenyl chloroformate or 2,2,2-trihaloethylchloroformate in the presence of an organic solvent. The organic solvent is preferably a halogenated hydrocarbon. The dealkylation process can proceed via the formation of corresponding carbamate intermediate, which need not be isolated from the reaction mixture in a solid form. The treatment of the carbamate intermediate with a base provides duloxetine. The base can be potassium hydroxide or sodium hydroxide. The duloxetine can be isolated from the reaction mixture as a free base or as a salt. The salt forms of duloxetine can be isolated by treating the free base of duloxetine with appropriate acid. Preferably the duloxetine is isolated as maleate or hydrochloride salt.

In another aspect of the present invention is provided a pharmaceutically acceptable salt of duloxetine having an enantiomeric purity of about 98% or above, for example about 99.5% or above, or about 99.9% or above. The pharmaceutically acceptable salt of duloxetine can be duloxetine maleate or duloxetine hydrochloride.

In yet another aspect of the present invention is provided a pharmaceutical composition comprising a pharmaceutically acceptable salt of duloxetine having an enantiomeric purity of about 99.5% or above, for example about 99.9% or above, and optionally containing one or more excipients. The pharmaceutically acceptable salt of duloxetine can be duloxetine maleate or duloxetine hydrochloride.

In still another aspect of the present invention is provided a method for inhibiting serotonin uptake in mammals which comprises administering to a mammal a pharmaceutically effective amount of a pharmaceutically acceptable salt of duloxetine having an enantiomeric purity of more than about 99.5%, for example about 99.9% or above. The pharmaceutically acceptable salt of duloxetine can be duloxetine maleate or duloxetine hydrochloride.

While the present invention has been described in terms of its specific embodiments, certain modifications and equivalents will be apparent to those skilled in the art and are intended to be included within the scope of the present invention.

### Example 1

### Preparation of Duloxetine Maleate

### a) Preparation of 3-dimethylamino-1-(2-thienyl)-1-propanaone hydrochloride

A mixture of 2-acetylthiophene (100 g), dimethylamine hydrochloride (81.21 g), paraformaldehyde (35.36 g) and concentrated hydrochloric acid (3.7 g) in isopropyl alcohol (240 mL) was stirred under reflux for 12 h. The mixture was cooled to 0-5° C. and stirred at the same temperature for 6 h. The solid was filtered, washed with cold (5-10° C.) isopropyl alcohol (100 mL), and dried under vacuum at 45-50° C. for 12 h to obtain the title compound as a white solid.

Yield: 135 g

### b) Preparation of 3-dimethylamino-1-(2-thienyl)-1-propanol

A solution of 1 N sodium hydroxide (460 mL) was added to a mixture of 2-thienyl-2-dimethylaminoethyl ketone hydrochloride (100 g) in methanol (350 mL) at about 25° C. to the pH of 11. After cooling the reaction mixture to 10-15° C., sodium borohydride (8.5 g) was added in portion over a period of 30 minutes. The reaction mixture was stirred at about 25° C. for 2 h. After the completion of the reaction, excess sodium borohydride was decomposed by addition of acetone at about 25° C. The mixture was stirred at about 25° C. for 30 minutes. After concentrating the reaction mixture under reduced pressure to about ⅓ of the initial volume, the concentrated reaction mixture was diluted with water (200 mL) and extracted with ethyl acetate (2×400 mL). The ethyl acetate layer was washed with water and concentrated under reduced pressure. Hexanes (200 mL) were added to the residue and the mixture was stirred for at about 25° C. for 1 h and at 5°-10° C. for a further hour. The solid was filtered and dried under vacuum at 40-45° C. for 4-6 h to provide the title compound as an off-white solid.

Yield: 78 g

### c) Preparation of (1S)-3-(dimethylamino)-1-(2-thienyl)propan-1-ol.(S)-(+)-mandelate

A solution of S-(+)-mandelic acid (34.4 g) in absolute ethanol (70 mL) was added to a mixture of racemic dimethylamino alcohol obtained from step (b) (70 g) in ethyl acetate (630 mL) at 45-50° C. over a period of 25 minutes. The mixture was stirred at 45-50° C. for 1 h and at about 25° C. for 12 h. The solid material precipitated out was filtered, washed with ethyl acetate (2×70 mL) and dried under vacuum at 55-60° C. for 4-6 h to obtain the title compound as a white solid.

Yield: 58 g

### d) Preparation of (1S)-3-(dimethylamino)-1-(2-thienyl)propan-1-ol

The Mandelic acid salt obtained from step (c) (58.4 g) was suspended in water and the pH of the suspension was adjusted to about 12 by the addition of a 1 N aqueous solution of sodium hydroxide at about 25° C. The reaction mixture was extracted with toluene (584 mL) and again with toluene (292 mL). After washing with water, toluene layer was concentrated under reduced pressure to obtain the title compound.

### e) Preparation of (1S)-N,N-dimethyl-3-(1-naphthalenyloxy)-3-(2-thienyl)propanamine

Sodium hydride (7.27 g) was added to the residue obtained from step (d) in dimethyl sulfoxide (122 mL) at 10-20° C. in portions over a period of 30 minutes. The mixture was stirred at about 25° C. for 30 minutes. Potassium benzoate (237 g, 0.0173 mole) was added to the reaction mixture and stirred at about 25° C. for 30 additional minutes. A solution of 1-fluoronaphthalene (30.3 g) in dimethyl sulfoxide (61 mL) was added to the reaction mixture at about 25° C. The reaction mixture was stirred at 50-55° C. for 3-4 h. After the completion of the reaction, the reaction mixture was cooled to 10° C. and acidified to the pH of 4-4.5 with acetic acid. The reaction mixture was diluted with water (732 mL) and further acidified to the pH of 1.5-2.0 with 6 N hydrochloric acid. The reaction mixture was washed with hexane (2×290 mL) at about 25° C. The pH of the aqueous layer was adjusted to about 11 using 30% aqueous sodium hydroxide solution and subsequently extracted with ethyl acetate (2×584 mL). The ethyl acetate layer was washed with water (2×584 mL) and concentrated under reduced pressure to obtain the title compound as an oily mass.

### f) Preparation of (S)-N-methyl-N-phenoxycarbonyl-3-(1-naphthalenyloxy)-3-(2-thienyl)propanamine

Phenyl chloroformate (30.82 g) was added to the mixture of residue obtained from example e) and diisopropylethylamine (4.07 g) in chloroform (497 mL) at 10°-15° C. The reaction mixture was stirred at about 25° C. for 2 h. After the completion of the reaction, 1% aqueous sodium bicarbonate solution (584 mL) was added to the reaction mixture and stirred at 45-50° C. for 1 h. The mixture was cooled to about 25° C. and the layers were separated. The organic layer was successively washed with 0.5 N hydrochloric acid solution (350 mL), 1% sodium bicarbonate (292 mL) and water (2×350 mL). The solvent was evaporated under reduced pressure to obtain the title compound as an oily mass.

### g) Preparation of (S)-N-methyl-3-(1-naphthalenyloxy)-3-(2-thienyl) propanamine

Pulverized potassium hydroxide (68 g) was added to the residue obtained from step (f) in toluene (467 mL). The reaction mixture was stirred under reflux for 6 to 8 h. After the completion of the reaction, the reaction mixture was cooled to about 25° C. The solid residue was filtered and washed with toluene (3×147 mL). The toluene layer was washed with water (2×467 mL) to adjust the pH to between about 7 and about 8 and concentrated under reduced pressure to obtain the title compound as an oily mass.

### h) Preparation of Duloxetine Maleate

Maleic acid (16.37 g) was added to the residue obtained from step g) in ethyl acetate (496 mL) at 40-45° C. The reaction mixture was stirred at 45-50° C. for 1 h and further at room temperature for 4 h. The solid was filtered, washed with ethyl acetate (2×87.5 mL) and dried in air at 45-50° C. for 6 to 8 h to obtain the title compound as a cream colored solid.

Yield: 42 g

Chemical Purity: 99.47%

Enantiomeric Purity: 99.98%

1. A pharmaceutically acceptable salt of duloxetine having an enantiomeric purity of about 98% or above.

2. A pharmaceutically acceptable salt of duloxetine having an enantiomeric purity of about 99.5% or above.

3. A pharmaceutically acceptable salt of duloxetine having an enantiomeric purity of about 99.9% or above.

4. The pharmaceutically acceptable salt of duloxetine according to any one of claims 1 to 3 wherein is the salt is the maleate or the hydrochloride.

5. A pharmaceutically acceptable salt of a compound of Formula I having an enantiomeric purity of about 98% or above prepared by using a process comprising, 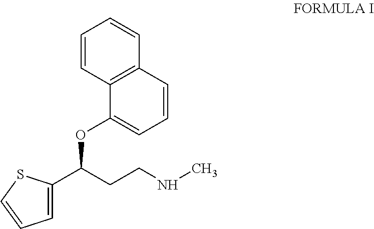
a) reacting (1S)-3-(dimethylamino)-1-(2-thienyl)propan-1-ol of Formula or its salts, 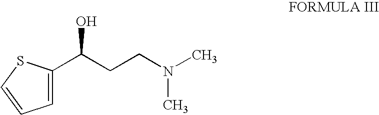
with 1-fluoronaphthalene in the presence of an organic solvent to obtain (3S)-N,N-dimethyl-3-(1-naphthyloxy)-3-(2-thienyl)propan-1-amine of Formula II or its salts, 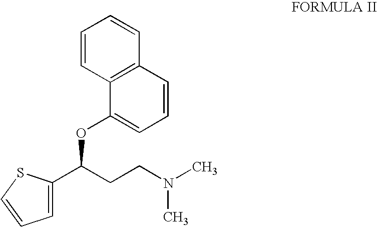
dealkylating (3S)-N,N-dimethyl-3-(1-naphthyloxy)-3-(2-thienyl)propan-1-amine of Formula II or its salts obtained from step a) to obtain duloxetine of Formula I or its salts, and
c) isolating duloxetine of Formula I or its salts from the reaction mixture thereof.

6. The pharmaceutically acceptable salt of claim 5 wherein the process for its preparation does not involve the isolation of the compound of Formula II from the reaction mixture in any solid form.

7. The pharmaceutically acceptable salt of claim 5, where in the salt is maleate or hydrochloride.

8. A process for preparing a compound of Formula I having an enantiomeric purity of about 98% or above comprising 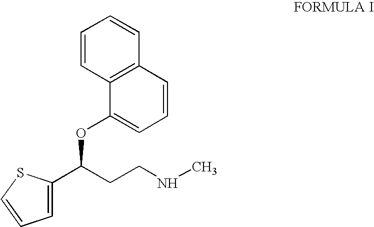
a) reacting (1S)-3-(dimethylamino)-1-(2-thienyl)propan-1-ol of Formula III or its salts, 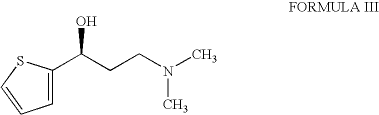
with 1-fluoronaphthalene in the presence of an organic solvent to obtain (3S)-N,N-dimethyl-3-(1-naphthyloxy)-3-(2-thienyl)propan-1-amine of Formula II or its salts, 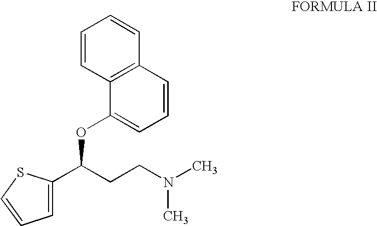
b) dealkylating (3S)-N,N-dimethyl-3-(1-naphthyloxy)-3-(2-thienyl)propan-1-amine of Formula II or its salts obtained from step a) to obtain duloxetine of Formula I or its salts, and
c) isolating duloxetine of Formula I or its salts from the reaction mixture thereof.

9. The process of claim 8 wherein the compound of Formula II is not isolated from the reaction mixture in any solid form.

10. A process according to claims 8, wherein the organic solvent is at least one of dimethylsulfoxide, C₁₋₃ alkanol, toluene, chloroform, dioxane, dimethylformamide, dimethylacetamide or tetrahydrofuran.

11. A process according to claim 8, wherein the organic solvent is dimethylsulfoxide or dimethylacetamide.

12. A process according to claim 8, wherein step (b) is carried out in the presence of phenyl chloroformate or 2,2,2-trihaloethylchloroformate.

13. A process according to claim 8, wherein duloxetine of Formula I is isolated as duloxetine maleate.

14. A process according to claim 8, wherein duloxetine of Formula I is isolated as duloxetine hydrochloride.

15. A pharmaceutical composition comprising the salts of claim 5.

16. A pharmaceutical composition comprising the maleate or hydrochloride salt of claim 7.

16. A method for inhibiting serotonin uptake in mammals which comprises administering a pharmaceutically effective amount of salts of claim 5.

17. A method for inhibiting serotonin uptake in mammals which comprises administering a pharmaceutically effective amount of the maleate or hydrochloride salt of claim 7.
